(19) United States
(12) Patent Application Publication
(10) Pub. No.: US 2008/0234580 A1
(43) Pub. Date: Sep. 25, 2008

(54) Ultrasonic Imaging of Perfusion and Blood Flow with Harmonic Contrast Agents

(75) Inventors: Matthew Bruce, Seattle, WA (US); Jeffrey E. Powers, Bainbridge Island, WA (US); David Hope Simpson, Bothell, WA (US); Michalakis Averkiou, Kirkland, WA (US)

Correspondence Address: PHILIPS MEDICAL SYSTEMS;PHILIPS INTELLECTUAL PROPERTY & STANDARDS, P.O. BOX 3003, 22100 BOTHELL EVERETT HIGHWAY, BOTHELL, WA, 98041-3003, US

(73) Assignee: KONINKLIJKE PHILIPS ELECTRONICS, N.V.

(21) Appl. No.: 10/597,532

(22) PCT Filed: Jan. 31, 2005
(86) PCT No.: PCT/IB2005/050404

§ 371 Date: Jul. 28, 2006

Related U.S. Application Data
(60) Provisional application No. 60/542,259, filed on Feb. 5, 2004.

(51) Int. Cl. A61B 8/00; A61B 8/14

(52) U.S. Cl. 600/441; 600/458; 600/447

(57) ABSTRACT

An ultrasonic diagnostic imaging method and system produce diagnostic contrast images depicting both tissue perfusion and flow velocity in larger vessels by utilizing both linear and nonlinear imaging techniques. A sequence of echoes from differently modulated transmit pulses is received and processed in different ways to detect nonlinear signals from microbubble-perfused tissue and Doppler blood flow in larger vessels. The Doppler flow signals may be either linear or nonlinear or a mixture of both. A decision circuit classifies the detected signals for display pixels in a perfusion and/or flow and/or tissue image. Separate perfusion and flow images can be simultaneously displayed or an image of both perfusion and flow can be displayed.

This invention relates to ultrasonic diagnostic imaging systems and, in particular, to the use of ultrasonic imaging to segment and visualize perfusion of tissue and blood vessel flow with ultrasonic contrast agents.

Ultrasonic diagnostic imaging has benefited from the enhancement of perfusion studies and blood flow imaging with harmonic contrast agents for a number of years. In a typical study the contrast agent is introduced into the patient intravenously. Ultrasonic imaging is then commenced at a region of interest such as the heart or blood vessels. As the injected bolus of contrast agent begins arriving at the region of interest the microbubbles of the contrast agent return relatively strong ultrasonic echoes. Furthermore, these echo signals have significant nonlinear (e.g., second harmonic) components. Detecting signals at the second harmonic of the transmit frequency thus produces signals from the contrast agent which dominate those returned by other reflectors in the body. An image which maps the locations of the contrast agent in the body thus reveals the locations of the blood flow which carries the microbubbles, and images produced from the second harmonic signals and other harmonic components segment out the locations of blood flow to the relative exclusion of the surrounding tissue.

The use of contrast agents to image the perfusion of microvasculature in tissues such as the myocardium or liver has been found to produce excellent results which enable various techniques for quantifying the perfusion of tissue with a flow of blood. As used herein, the term “perfusion” relates to the amount of blood flow per volume of tissue. With the advent of low power (low MI) contrast agents and techniques, a single dose of contrast agent can provide a relatively long period during which the contrast agent is present in the body and perfusing the tissue. However, such long imaging periods are generally not prevalent when imaging and diagnosing larger blood vessels. The larger arterial blood vessels will usually begin to fill first following the bolus injection of the contrast agent and can initially be imaged with good results. But in time the contrast agent will begin to fill the microvasculature of the surrounding tissue, obscuring the flow of contrast agents in the larger vessels. One technique for dealing with this problem is to image at a higher MI which is just high enough to continuously destroy the slower moving microbubbles in the microvasculature of the region of interest while continuing to visualize the faster moving microbubbles in the larger vessels. This approach will reduce the effective imaging time because the contrast agent is constantly being destroyed. Furthermore, it is often difficult to adjust and maintain the appropriate transmit power levels in a clinical environment to maintain the constant destruction of microvasculature microbubbles without significantly disrupting those in the larger vessels. It would be desirable to be able to image both contrast agent tissue perfusion and the flow of contrast agents in the larger vessels simultaneously as both provide significant useful clinical information. But it is desirable to do so in a way which affords substantial contrast agent imaging time to enable the most comprehensive and accurate diagnosis of the patient.

In accordance with the principles of the present invention, an ultrasonic diagnostic imaging system and method are described which provide the simultaneous imaging of both tissue perfusion and larger vessel blood flow by contrast agents. Multiple, differently modulated transmit signals are used to acquire ensembles of echo information from points within a region of interest. The ensembles are differently filtered to produce contrast agent signals characteristic of perfusion and/or higher velocity blood flow. The ultrasound system uses these signals to produce an image of the blood flow in larger vessels against a background of tissue perfusion. The clinician is thus able to visualize tissue perfusion and larger vessel flow simultaneously with the larger vessel flow clearly segmented against the perfusion background.

In the drawings:

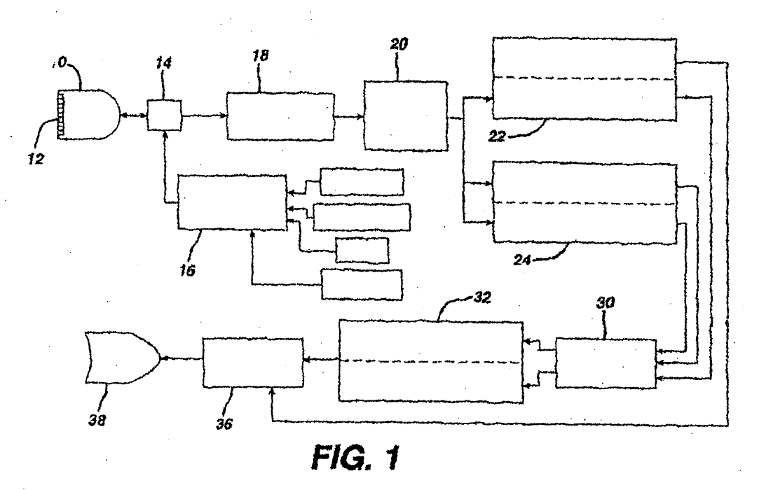

FIG. 1 illustrates in block diagram form an ultrasound system constructed in accordance with the principles of the present invention.

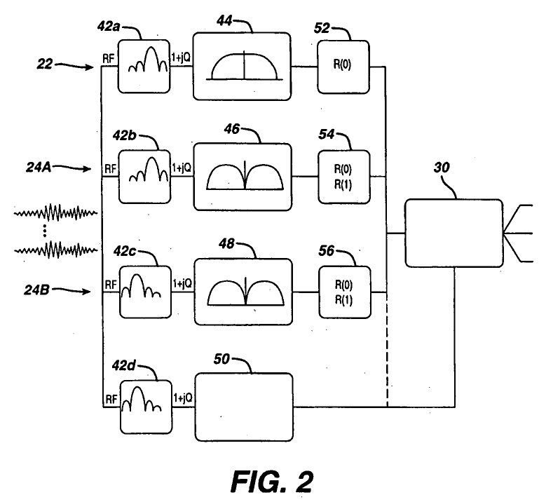

FIG. 2 is a detailed block diagram of the contrast signal filtering of the detection and classification of ultrasound signals from different sources in the ultrasound system of FIG. 1.

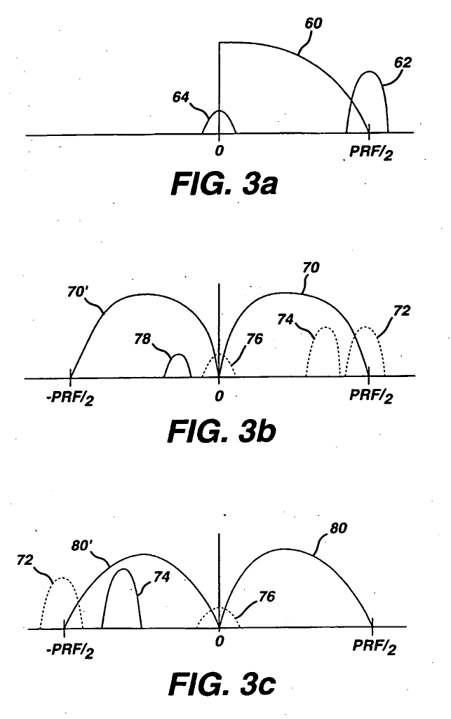

FIGS. 3a-3c illustrate the characteristics of the filters of FIG. 2.

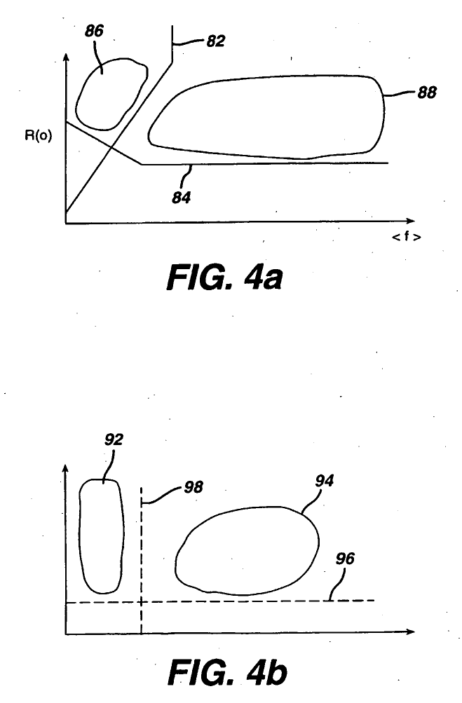

FIG. 4a-4b illustrate response characteristics useful for classifying the received signals in the embodiment of FIG. 2.

Referring first to FIG. 1, an ultrasonic diagnostic imaging system constructed in accordance with the principles of the present invention is shown in block diagram form. This system operates by scanning a two or three dimensional region of the body being imaged with ultrasonic transmit beams. As each beam is transmitted along its steered path through the body, the tissue and blood flow in the body return echo signals with linear and nonlinear (fundamental and harmonic) components corresponding to the transmitted frequency components. The transmit signals are modulated by the nonlinear effects of the tissue through which the beam passes or the nonlinear response of a contrast agent microbubble encountered by the beam, thereby generating echo signals with nonlinear components.

The ultrasound system of FIG. 1 utilizes a transmitter 16 which transmits waves or pulses of a selected modulation characteristic in a desired beam direction for the return of harmonic echo components from scatterers within the body. The transmitter is responsive to a number of control parameters which determine the characteristics of the transmit beams as shown in the drawing, including the frequency components of the transmit beam, their relative intensities or amplitudes, and the phase or polarity of the transmit signals. The transmitter is coupled by a transmit/receive switch 14 to the elements of an array transducer 12 of a probe 10. The array transducer can be a one dimensional array for planar (two dimensional) imaging or a two dimensional array for two dimensional or volumetric (three dimensional) imaging.

The transducer array 12 receives echoes from the body containing fundamental and harmonic (nonlinear) frequency components which are within the transducer passband. These echo signals are coupled by the switch 14 to a beamformer 18 which appropriately delays echo signals from the different transducer elements, then combines them to form a sequence of fundamental and harmonic signals along the beam from shallow to deeper depths. Preferably the beamformer is a digital beamformer operating on digitized echo signals to produce a sequence of discrete coherent digital echo signals from a near field to a far field depth of field. The beamformer may be a multiline beamformer which produces two or more sequences of echo signals along multiple spatially distinct receive scanlines in response to a single transmit beam, which is particularly useful for 3D imaging. The beamformed echo signals are coupled to an ensemble memory 20.

In accordance with one aspect of the present invention, multiple waves or pulses are transmitted in each beam direction using different modulation techniques, resulting in the reception of multiple echoes for each scanned point in the image field. The echoes corresponding to a common spatial location are referred to herein as an ensemble of echoes, and are stored in the ensemble memory 20, from which they can be retrieved and processed together. The echoes of an ensemble are processed in various ways as described more fully below to produce the desired fundamental or harmonic signals. The echo signals are processed by a B mode signal path including a grayscale signal processor 22 and by a Doppler signal path including a Doppler processor 24. In the illustrated embodiment the Doppler processor is provided in an ASIC (application specific integrated circuit) which includes two parallel paths for the processing of two Doppler signals at the same time. These paths are shown as Doppler processor A and Doppler processor B in the drawing.

The grayscale and Doppler processors can be operated individually in the conventional manner to produce a grayscale image or a Doppler image, or a colorflow image which is formed by the overlay of a fundamental or harmonic grayscale tissue image with Doppler flow information. In accordance with the present invention signals from the grayscale and Doppler processors 22 and 24 are coupled to a classifier 30. In a constructed embodiment the classifier is formed by software running on a CPU which analyzes the received signals and decides whether a received signal should be displayed as a pixel in a flow image or a pixel in a perfusion image or both. For example a large vessel may be visualized in both a perfusion image and a flow image. The signal is appropriately stored in an image memory 32 which is partitioned into a flow image section and a perfusion image section. The flow and perfusion images are further processed as by scan conversion and combined in an overlay of the flow image overlaying the perfusion image by an image processor 36. Alternatively the flow information can be embedded in the perfusion image in the image memory. Additionally the flow and perfusion images can overlay an image of the tissue background. The resultant image is displayed on an image display 38.

The vascular flow and/or perfusion images may be alternatively removed via a user control either in review of a Cineloop sequence or during live imaging. This enables the clinician to view the perfusion image over tissue, the flow image over tissue or both tissue and flow together over tissue. It also enables either perfusion or flow to be viewed in isolation from other information. The transparency of the perfusion and flow images can be altered to enable visualization of perfusion, flow and background tissue images together. For example, when the microcirculation is filled with microbubbles the perfusion image may largely obscure underlying tissue or flow. The perfusion may then be displayed in a semi-transparent mode so that the clinician can view the underlying tissue or flow while still appreciating the tissue perfusion.

In use, an ultrasonic contrast agent is introduced into the patient's vascular system and imaging of a region of interest such as the liver commences at a low MI. Initially, before the contrast agent arrives at the region of interest, a tissue image is formed by signals received from tissue. These signals are processed by a background tissue signal processor in the B mode signal path. The background tissue signal processor produces an image of the background tissue in the region of interest in a manner similar to the processing of the grayscale signal processor 22, but with thresholds set to detect signals from tissue. The tissue signals may be fundamental or harmonic. Generally fundamental signals are preferred when operating at a low MI where tissue harmonic signals will be at low levels. The background tissue image is coupled to the image processor 36 where it is displayed initially as just a tissue image, then as a background to flow and perfusion as the contrast agent begins to fill the region of interest.

The larger arterial vessels in the region of interest will begin to light up first in the image as the contrast agent will arrive in the larger vessels first due to their higher flow velocities. The larger vessels are displayed from the fundamental and harmonic signals produced by flowing contrast agent and detected by the Doppler processor 24. In time, the contrast agent will begin to perfuse the tissue surrounding the larger vessels and the microbubbles slowly begin to perfuse the microvasculature of the tissue. This filling of the microvasculature with microbubbles increases the nonlinear signals detected by the grayscale signal processor. This perfusion of the tissue will then light up from the nonlinear (harmonic) amplitude response of the signals produced by the grayscale signal processor. The displayed image will thus appear as a contrast perfusion image containing larger vessels of more rapidly flowing contrast microbubbles. The signal paths A and B of the Doppler processor 24 are operated at the harmonic and fundamental frequencies respectively. In a constructed embodiment this fundamental and nonlinear mixing enables the display of the larger vessels in the near field as nonlinear (harmonic) contrast segments and the larger vessels in the far field as linear fundamental contrast segments, thereby compensating for the attenuation of higher harmonic frequencies from the deeper depths. The fundamental and harmonic signals may be blended together into one flow image, thereby showing larger vessel flow over a considerable depth of field against a background of perfused tissue.

A variety of different transmit sequences may be employed to detect the nonlinear signal components. Harmonic separation is preferably performed by what is known as “pulse inversion,” by which the echoes from multiple, differently modulated transmit pulses are combined to separate the harmonic components and attenuate linear fundamental components. The different modulation may be different phase modulation, polarity modulation, or amplitude modulation, or a combination thereof as described in U.S. patent application Ser. No. 60/527,538, filed Dec. 5, 2003 and entitled “ULTRASONIC SPECKLE REDUCTION USING NONLINEAR ECHO COMBINATIONS.” For example, a transmit sequence may comprise three transmit pulses transmitted at the desired pulse repetition interval (PRI) for the Doppler flow velocities being detected, with the first pulse having a nominal amplitude of 0.5 and a phase or polarity of 0° or +, a second pulse having a nominal amplitude of 1.0 and a phase or polarity of 180° or −, and a third pulse having a nominal amplitude of 0.5 and a phase or polarity of 0° or +. When the echoes from the three transmit pulses are normalized in amplitude and additively combined the linear fundamental frequency components cancel and the harmonic components are enhanced. With amplitude and phase modulation (see U.S. Pat. No. 6,095,980 (Burns et al.) and U.S. Pat. No. 6,319,203 (Averkiou)) it is possible to combine the nonlinear echoes in a way that some energy at the fundamental frequency is present. This energy is referred to herein as “nonlinear fundamental” energy and it is the result of the harmonic activity at unequal amplitudes. The “nonlinear fundamental” should not be confused with the linear response of a system with a single excitation at the fundamental frequency. The nonlinear fundamental can be used in both the detection of perfusion and/or the detection of vascular flow. Alternatively, pairs of differently modulated pulses transmitted at a rapid rate to prevent motion artifacts can be transmitted at the PRI from one pulse pair to the next as described in U.S. Pat. No. 6,620,103 (Bruce et al.), which shows how the pulse pairs can be spatially interleaved over different transmit lines for low velocity flow detection.

A detailed diagram of the grayscale and Doppler processors is shown in FIG. 2. The echo signals from an ensemble of transmit pulses 1-N are applied to the inputs of the processors. The grayscale signal path 22 includes a quadrature bandpass filter (QBP) 42, which passes harmonic echo signals in a band around 2fₒ. Construction and operation of a quadrature bandpass filter is described in U.S. Pat. No. 6,050,942 (Rust et al.) which explains how the filter produces the I and Q components of an echo signal in a desired passband. These components of an echo ensemble are further filtered by the lowpass characteristic of a matrix wall filter, which passes echoes returned from tissue and stationary microbubbles. The matrix wall filter may exhibit a response characteristic such as that illustrated in FIG. 3a. The low pass filter response is seen to roll off at PRF/2, half the repetition frequency of the echo ensemble. For the case of pulse inversion, fundamental frequency echo signals from tissue or stationary microbubbles are contained in a band 62 located at the end of the passband, since the QBP is operating in the harmonic band. Harmonic signal components are located in a band 64 at the nominal DC point of the filter response. The filtered ensemble of echoes is applied to an estimator 52 which combines the echo signals to separate the nonlinear second harmonic signal components and detects the signal power or amplitude squared. This signal path functions in the manner of a nonlinear pulse inversion processor to pass nonlinear signals from stationary or nearly stationary microbubbles which have perfused tissue in the region of interest.

The second signal path 24A in this embodiment is a Doppler processing path which includes a QBP 42b set to pass harmonic frequencies. This could be the same QBP as used in the grayscale signal path or could be a separate QBP as shown in the drawing. The signals passed by QBP 42b are filtered by a bandpass matrix wall filter 46. This filter is designed to detect harmonic flow signals as there is considerable overlap of fundamental and harmonic components produced by the QBP when broadband transmit pulses are used. Nonlinear pulsing schemes other than phase or polarity modulated pulse inversion (e.g., combinations of phase or polarity pulse inversion and amplitude modulation) may be used in a similar fashion to detect both perfusion and flow of contrast agents and to separate linear and nonlinear components even in the case of broadband transmit signals. A response characteristic useful for this first Doppler signal path is shown in FIG. 3b. The bandpass characteristic 70, 70′ is seen to have a stop band at DC. The fundamental frequencies from microbubbles are located as shown at 72, and at 74 for moving microbubbles, attenuated by the QBP 42b. Harmonic signal components from stationary microbubbles are located in a band 76 at the stop band of the filter, and detectable signals from moving microbubbles are located in the band 78. The filtered harmonic flow signal ensemble is coupled to an estimator 54 which estimates the harmonic flow signals. These signals will exhibit good axial and lateral resolution due to the high harmonic frequencies and will exhibit good signal-to-clutter ratios since the echoes from flowing microbubbles are significantly stronger than returned tissue harmonic signals. These echoes will thus provide good spatial resolution by comparison with the blooming effect which can be seen with fundamental frequency flow detection. Further details of harmonic Doppler processing can be found in U.S. Pat. No. 6,036,643 (Criton et al.), which is in the context of tissue harmonic Doppler.

The third signal path 24B includes a QBP 42c set to pass fundamental frequencies fₒ. The fundamental frequency ensemble passed by this QBP is filtered by a matrix wall filter 48 with a band pass characteristic such as that shown in FIG. 3c. Harmonic components in the vicinity of the stop band 76 are attenuated, aided by the QBP response and depth-dependent attenuation, as are echo signals from stationary components in band 72 at the filter skirt. Moving microbubbles will exhibit a relatively strong response in band 74. These echo signal components will have a good signal-to-noise ratio in comparison to harmonic signals, but will have a lower signal-to-clutter response and exhibit relatively low spatial resolution since strong fundamental frequency echo signals are returned by both tissue and microbubbles. Image segments using these echoes can exhibit some blooming due to the high sensitivity of detection and signal-to-noise ratio of the Doppler signal estimator 56. Consequently, in a preferred embodiment, flow in the near field utilizes the harmonic Doppler flow signals produced by path 24A. At deeper depths where the harmonic frequencies become attenuated, the fundamental Doppler flow signals are used for display. At intermediate depths the flow display is a blend of the harmonic and fundamental signals, making a varying ratio transition from 100% harmonic to 100% fundamental over a transition depth region. The blending of harmonic and fundamental signals with depth is described in U.S. Pat. No. 6,283,919 (Roundhill et al.) Thus, flow can be shown in the perfusion and flow image over a considerable depth of view.

The signals produced by the three processing paths 22, 24A, and 24B are classified for use as tissue, stationary microbubbles (perfusion) or flowing microbubbles (in larger vessels) by the classifier 30. This classification could be done on the basis of the power and velocity estimates of the wall filtered signals. In the illustrated embodiment the classifier decides the image(s) in which to display the echoes in response to a velocity variance estimation path which includes a QBP 42d operating at the fundamental frequency fₒ, and a velocity variance processor 50. As shown by the dashed line at the output of the velocity variance processor, these variance estimates may also be classified and used for display. The variance estimate is computed as the root mean square of the Doppler velocity bandwidth, which, to a first order approximation, can be estimated using an equation of the form

$$1 - \frac{\vert R ( 1 ) \vert}{\vert R ( o ) \vert} ,$$

where R(o) is the echo value squared and can be computed by

$$R ( o ) = \frac{1}{N} {\sum }_{n = 1}^{N} x ( n ) {x}^{*} ( n )$$

and R(1) is the magnitude of the first lag of the autocorrelation of the echo values and can be computed by

$$R ( 1 ) = \frac{1}{N} {\sum }_{n = 1}^{N} x ( n ) {x}^{*} ( n - 1 ) .$$

Using velocity for signal segmentation is premised on the assumption that tissue and microbubbles in microvasculature (perfusion) move at relatively low velocities and exhibit a relatively narrow dispersion of velocities, and microbubbles in larger vessels move at relatively higher velocities and exhibit a relatively wider dispersion of velocities. A segmentation scheme such as that shown in FIG. 4a can be used by the classifier 30 in response to calculations of mean velocity. This scheme has two curves 82 and 84 which divide the response into different regions. If, for instance, a velocity estimate R(o) for a pixel exhibits relatively low mean velocity (Doppler frequency) <f> and relatively high power, it is likely that the signal came from tissue and it will be displayed in the perfusion image. These velocity estimates will be in the region defined generally by the area 86. Those signals exhibiting higher mean velocities <f> and relatively lower power are likely from flow, and signals classified generally in the area 88 will be displayed in the Doppler flow image.

Another classification scheme which is predicated on variance estimates is shown in FIG. 4b. Estimates R(o) with relatively narrow (low) velocity variance and relatively significant power in area 92 are likely lower velocity perfusion microbubbles in the microvasculature of tissue and will be displayed in the perfusion image. Estimates R(o) which have somewhat less power but exhibit a broader (higher) velocity variance in area 94 are likely from flowing microbubbles and will be displayed in the flow image. Signals below the variance threshold 98 are classified as from tissue with or without microbubbles in its microcirculation. Signals below the amplitude or power threshold 96 are classified as noise which exhibits broad, random variance.

Thus it is seen that both perfusion and blood flow can be segmented and imaged simultaneously for an extended period of time in a single displayed image by imaging contrast at low MIs and utilizing the concepts of the present invention.

What is claimed is:

1. A method of ultrasonically imaging blood perfusion and blood flow in a region of interest of a body comprising:
acquiring a sequence of ultrasonic echo signals from a body which has been infused with an ultrasonic contrast agent;
processing the echo signals to detect the tissue structure in the absence of microbubbles;
processing a plurality of the echo signals in a first way to detect echo signals returned from tissue microvasculature perfused with the contrast agent;
processing a plurality of the echo signals in a second way to detect echoes returned from blood flow containing the contrast agent in larger vessels;
utilizing the echo signals processed the first way to form a portion of an image depicting perfusion;
utilizing the echo signals processed the second way to form a portion of an image depicting blood flow in larger vessels; and
displaying an ultrasound image depicting both contrast-enhanced perfusion and contrast-enhanced blood flow.

2. The method of claim 1, wherein displaying further comprises depicting both the presence and locations of microbubbles in tissue and the velocity of microbubbles in blood flow.

3. The method of claim 1, further comprising deciding the portion of the image which an echo signal is to form on the basis of a blood flow velocity estimation.

4. The method of claim 3, wherein deciding further comprises deciding the portion of the image which an echo signal is to form on the basis of a blood flow variance estimation.

5. The method of claim 1, wherein processing a plurality of echo signals in first and second ways comprises processing the same ensemble of echo signals in first and second ways.

6. The method of claim 1, wherein acquiring a sequence of ultrasonic echo signals further comprises acquiring an ensemble of echoes over time from each of a plurality of different locations in the body.

7. The method of claim 1, wherein processing a plurality of the echo signals in a first way comprises detecting the amplitude or power of the echo signals; and
wherein processing a plurality of the echo signals in a second way comprises Doppler processing the plurality of the echo signals.

8. The method of claim 7, wherein processing a plurality of the echo signals in both the first way and the second way both include detecting nonlinear components of the echo signals by the pulse inversion technique.

9. The method of claim 1, wherein utilizing the echo signals processed the first way further comprises forming a perfusion image; and
wherein utilizing the echo signals processed the second way further comprises forming a flow image; and
wherein displaying an ultrasound image further comprises displaying the perfusion image overlaid with the flow image.

10. The method of claim 1, further comprising transmitting a plurality of differently modulated transmit pulses in each of a plurality of different beam directions;
wherein processing a plurality of the echo signals in both the first way and the second way both include detecting harmonic components of the echo signals by the pulse inversion technique.

11. An ultrasonic diagnostic imaging system for imaging both perfusion and flow in a body infused with a contrast agent comprising:
an ultrasonic transducer array operated to transmit a plurality of pulses in each of a plurality of different beam directions and to receive echoes in response to the pulses;
a beamformer coupled to the transducer array;
a first processor coupled to the beamformer and responsive to pluralities of echo signals for detecting echoes returned from perfused tissue;
a second processor coupled to the beamformer and responsive to ensembles of echo signals for detecting echoes returned from blood flow containing contrast in larger vessels;
a decision processor, coupled to the first and second processors, for identifying signals to be displayed on the basis of velocity;
an image memory responsive to the decision circuit which acts to utilize signals produced by the first and second processors to form a perfusion image portion and a flow image portion; and
a display coupled to the image memory which displays an ultrasound image which depicts both contrast perfused tissue and the flow in larger vessels in a common image.

12. The ultrasonic diagnostic imaging system of claim 11, wherein the second processor includes a first signal path which Doppler processes nonlinear echo ensembles and a second signal path which Doppler processes fundamental frequency echo ensembles,
wherein the display displays an image of nonlinear Doppler processed flow in the near field and fundamental frequency Doppler processed flow in the far field.

13. The ultrasonic diagnostic imaging system of claim 11, further comprising a transmitter, coupled to the transducer array, which acts to transmit a plurality of differently modulated beams in each of a plurality of different beam directions.

14. The ultrasonic diagnostic imaging system of claim 13, wherein each of the first and second processors process harmonic signals separated by the pulse inversion technique.

15. The ultrasonic diagnostic imaging system of claim 11, wherein the decision processor acts to identify signals to be displayed on the basis of velocity variance.

16. The ultrasonic diagnostic imaging system of claim 15, further comprising a velocity variance estimator responsive to echo signals processed by the first and second processors and coupled to the decision processor.

17. The ultrasonic diagnostic imaging system of claim 11, wherein the image memory comprises a first image buffer for storing a perfusion image and a second image buffer for storing a flow image.

18. The ultrasonic diagnostic imaging system of claim 11, further comprising a tissue signal processor which acts to detect echoes from tissue in the absence of microbubbles.

19. The ultrasonic diagnostic imaging system of claim 18, wherein the display acts to selectively display an image which is less than all of the combination of a tissue image component, a perfusion image component, and a flow image component.

20. The ultrasonic diagnostic imaging system of claim 19, further comprising means for adjusting the opacity of one of the image components to be semi-transparent, whereby obscured tissue or flow may be visualized through the semi-transparent image component.
